(19) United States
(12) Patent Application Publication
(10) Pub. No.: US 2017/0184564 A1
(43) Pub. Date: Jun. 29, 2017

(54) SENSOR FOR NITRIC OXIDE DETECTION

(71) Applicant: CASE WESTERN RESERVE UNIVERSITY, Cleveland, OH (US)

(72) Inventors: Chung-Chiun Liu, Cleveland, OH (US); Laurie Dudik, Cleveland, OH (US); Freda Li, Cleveland, OH (US)

(21) Appl. No.: 15/127,332

(22) PCT Filed: Mar. 19, 2015
(86) PCT No.: PCT/US15/21533

§ 371 Date: Sep. 19, 2016

Related U.S. Application Data
(60) Provisional application No. 61/955,268, filed on Mar. 19, 2014.

(51) Int. Cl. G01N 33/497; G01N 33/00

(52) U.S. Cl. CPC G01N 33/497; G01N 33/0037; G01N 2033/4975; A61B 5/082

(57) ABSTRACT

A sensor for detecting nitric oxide includes a substrate, a working electrode formed on a surface of the substrate, a counter electrode formed on the surface of the substrate, a dielectric layer covering a portion of the working electrode and counter electrode and defining an aperture exposing other portions of the working electrode and counter electrode, a polyelectrolyte film covering the exposed other portions working electrode and counter electrode, the polyelectrolyte film including at least one metalloporphyrins compound or metallophthalocyanine compound capable of increasing the rate of electrochemical oxidation-reduction reaction with nitric oxide and providing the detection of nitric oxide at a lower oxidation potential.

### RELATED APPLICATION

This application claims priority from U.S. Provisional Application No. 61/955,268, filed Mar. 19, 2014, the subject matter of which is incorporated herein by reference in its entirety.

### BACKGROUND

Nitric oxide is one of the most extensively investigated molecules in the fields of inorganic and bioinorganic chemistry. The study of the molecule in biological systems received a renewed interest because of its role in a myriad of biological events. It is probably correct to state that nitric oxide is involved in practically every common pathophysiological event by virtue of its importance in the normal maintenance of many important physiological phenomena ranging from the protection of the heart, stimulation and regulation of brain functions and vascular tone, to responding to vascular injuries and pulmonary diseases. The 1998 Nobel Prize in Medicine was awarded jointly to Robert F. Fuchogott, Louise J. Ignarro and Ferid Murad for their discoveries concerning “Nitric Oxide as a Signaling Molecule in the Cardiovascular System”.

The production of NO in the human body proceeds via one of two pathways: an enzymatic and a nonenzymatic pathway. The enzymatic pathway involves the action of the nitric oxide synthases (NOS) on the amino acid arginine with the production of the metabolites citrulline and NO. This five-electron oxidation reaction requires reduced pyridine nucleotides, reduced biopteridines and calmodulin. In the bloodstream, NO binds primarily to hemoglobin, being then converted to NO₃⁻ and eliminated in the urine with a half-life of 5 to 8 hours.

NO₃⁻ from food and inhaled NO is concentrated in the saliva and converted to nitrite by bacteria on the surface of the tongue. When saliva is swallowed, the nitrite is converted to NO in the stomach, providing defense against swallowed microorganisms. This NO production is demonstrated in the stomach, on the surface of the skin, in infected nitrite-containing urine and in the ischemic heart.

Since the formation of NO is connected with several pathophysiological events, the measurement of NO is important for the characterization of important biological functions during which a change in the measured levels of NO produced may indicate the existence of a disease or pathogenesis event. One example for such a phenomena is the measurable change in NO production in exhaled air during airway inflammation in asthma and other diseases. Measurements of exhaled nitric oxide (ENO) are regarded as a marker for the airway inflammations as the concentration of ENO is nearly tripled in the pathogenesis of asthma. Exhaled NO is not increased during bronchospasm in the absence of coexisting inflammation, and it serves to differentiate between the components of asthma and thereby helps to direct to the appropriate medication.

In addition to biological events, it is known that oxides of nitrogen (NOₓ) originating from motor vehicles, fossil fuel and power plants are major pollutants that affect human health and the ecology. Primary emissions are CO, NO and unburnt hydrocarbons. It wasn't until the 1990s that NO emissions from cars were recognized as the major cause of environmental pollution (Menil et al., 2000). Furthermore, the nitrogen oxides (NO₂ or NO) are a source of ozone, which causes an increase of smog in large cities. This process, which occurs via solar irradiation and photolytic decomposition of NO₂, is a source of acid rain.

Monitoring the emission of these pollutants, their transport in the atmosphere, and their degradation to second-generation pollutants is crucial. Direct monitoring of NO in the emissions of combustion engines requires a sensor capable of sustaining high temperatures, low concentrations of NO (100-1000 ppm) and corrosive medium containing oxygen and water vapor. Under these conditions, the nitrogen oxide (NOₓ) mixtures contain mainly NO.

The present monitoring techniques of nitrogen oxide mixtures are expensive, the measuring devices are bulky and their use is therefore unpractical and problematic. Efforts have been concentrated on developing many kinds of NOₓ sensors, such as electrochemical sensors which utilize solid electrolytes, thin film superconductor type sensors, semiconductor oxide type sensors using SnO₂, ZnO, WO₃, and TiO₂ oxide ceramics or thin films, etc. Using SnO₂ as sensing material, the concentration of gaseous NO was determined to levels as low as 10 ppm whereas with solid electrolytes only concentrations in the order of 10³ ppm NO were detectable.

Nitric oxide (NO) is a small, uncharged, paramagnetic molecule, existing in gas and liquid phases. In the gas phase, the molecule is stable, compared with a short half-life of between 5 and 15 seconds measured in biological media. Its diffusion constant in physiological is very similar to that in water. The solubility of NO in hydrophobic solvents is nine times greater than in aqueous solutions, which makes NO an excellent transmitter agent and inflictor of cellular damage, acting without the necessity of specific export mechanism, such as vascular secretion. NO reacts with oxygen species and metals to yield oxidized products such as nitrites and nitrates, NO₂⁻ and NO₃⁻, respectively.

Several methods for detection of NO in solution and in the gas phase have been developed in recent years for diagnostic or environmental purposes. The fact that NO is very reactive in biological tissues makes its direct quantification very complex and many measurements, therefore, relied on indirect methods, determining levels of NO metabolites such as nitrite and nitrate anions or NO precursors such as citrulline instead of NO itself.

For directly measuring NO levels in vivo, 1,2-diaminoanthraquinone (DAQ) was found suitable. It produces a red-fluorescent precipitate when in contact with NO. This compound was used to detect changes in NO levels in rat retinas after injury to the optic nerve.

In another method, quantification of citrulline instead of NO was pursued. However, levels of the amino acid in sera and urine are not good indicators of NO production. In cultured cells, the presence of citrulline is primarily due to NO synthase enzyme (NOS) activity. Measurements indicated that the citrulline levels were not stoichiometrically equivalent to total NO levels as measured by a series of different methods (Marzinzig et al., 1997).

Other methods for NO identification and quantification include electrochemical, fluorescent and transistor-based methods. In one of these methods, the NO is trapped by nitroso compounds or reduced hemoglobin forming stable species that can be quantified by EPR (electron paramagnetic resonance) with a detection limit of 1 μM (30 ppb). In another method NO levels in the gas phase are detected by reaction with ozone, producing chemilumiescence, with a detection limit of 20 nM (ppt concentration). Recent electrochemical methods offer the possibility to measure even lower concentrations of NO (at the pM limit) in intact tissues and single cells.

Presently existing NO sensors have been manufactured for bedside treatments in hospitals and medical laboratories for the purposes of treatment and/or diagnostics. These sensors are based on the above-mentioned methods of analysis and thus suffer from several basic disadvantages, such as low S/N ratios, cross sensitivity to other components in the test medium, expensive and time-consuming operational steps and inaccurate quantification of NO or its metabolites due to NO's short half-life.

### SUMMARY

Embodiments described herein relate to a sensor for detecting, identifying, quantifying, and/or determining the amount or level of nitric oxide (NO) in a sample, and particularly relates to a biosensor for detecting, identifying, quantifying, and/or determining the amount or level of nitric oxide (NO) in a biological or bodily sample, such as breath, blood, and other physiological fluids.

The sensor includes a substrate, a working electrode formed on a surface of the substrate, a counter electrode formed on the surface of the substrate, a dielectric layer covering a portion of the working electrode and counter electrode and defining an aperture exposing other portions of the working electrode and counter electrode. A polyelectrolyte film covers the exposed portions of the working electrode and counter electrode and includes at least one metalloporphyrin compound or metallophthalocyanine compound. The at least one metalloporphyrin compound or metallophthalocyanine compound is capable of increasing the rate of electrochemical oxidation-reduction reaction with nitric oxide and providing the detection of nitric oxide at a lower oxidation potential.

In some embodiments, the polyelectrolyte film is semi-permeable to allow passage of nitric oxide through the film and inhibit passage anions that interfere with nitric oxide detection. The polyelectrolyte film can include a mixture of a perfluorosulfonic acid polymer and the at least one metalloporphyrin compound or metallophthalocyanine compound. The perfluorosulfonic acid polymer can include nafion or a nafion blend.

In other embodiments, the at least one metalloporphyrin compound or metallophthalocyanine compound contains as central atoms a metal atom selected from Fe, Co, Ni, Zn, Mn, Cu, Ru, V, Pb, or Cr. The at least one metalloporphyrin compound or metallophthalocyanine compound can be provided in the polyelectrolyte film at an amount of about 0.01 to about 10% by weight of the polyelectrolyte film. In some embodiments, the at least one metalloporphyrin comprises nickel(II)poly-tetrakis(3-methoxy-4-hydroxy-phenyl) porhydrin.

In still other embodiments, the working electrode and the counter electrode include metalized films. For example, the working electrode and counter electrode can independently comprise gold, platinum, palladium, silver, carbon, alloys thereof, and composites thereof. The metalized films can be provided on the surface of the substrate by sputtering or coating the films on the surface and then laser ablating the films to form the working electrode and counter electrode.

In other embodiments, the sensor can include a reference electrode on the surface of the substrate. The dielectric can cover a portion of the reference electrode. The sensor can also include a measuring device for applying voltage potentials to the working electrode and counter electrode and measuring the current flow between the working electrode and counter electrode to determine the level of nitric oxide in a sample, such as a biological sample.

### BRIEF DESCRIPTION OF THE DRAWINGS

FIG. 1 is a schematic illustration of a biosensor in accordance with an aspect of the application.

FIG. 2 is a top plan view of an array of biosensors in a row manufactured by a screen-printing process and laser ablation process.

FIG. 3 illustrates a plot showing differential pulse of voltammetry (DPV) measurements of nitric oxide over the concentration range of 4-19 ppb levels with and without CO₂ present using a biosensor with a gold working and counter electrode and Ag/AgCl reference electrode in accordance with one embodiment. The legend on the right hand side indicates the DPV measurements. The CO₂ is supplied by hard breadth through the mouth of an adult.

FIG. 4 illustrates a plot showing differential pulse of voltammetry (DPV) measurements of nitric oxide over the concentration range of 4-19 ppb levels with and without CO₂ present using a biosensor with a platinum working and counter electrode and Ag/AgCl reference electrode in accordance with one embodiment. The legend on the right hand side indicates the DPV measurements. The CO₂ is supplied by hard breadth through the mouth of an adult.

FIG. 5 illustrates a plot showing differential pulse of voltammetry (DPV) measurements of nitric oxide in a gas over the concentration range of 4-25 ppb levels using a biosensor with a platinum working and counter electrode and Ag/AgCl reference electrode in accordance with one embodiment.

FIG. 5 illustrates a plot showing differential pulse of voltammetry (DPV) measurements of nitric oxide in PBS over the concentration range of 0-225 ppb levels using a biosensor with a platinum working and counter electrode and Ag/AgCl reference electrode in accordance with one embodiment. The legend on the right hand side indicates the DPV measurements.

### DETAILED DESCRIPTION

Unless specifically addressed herein, all terms used have the same meaning as would be understood by those of skilled in the art of the subject matter of the application. The following definitions will provide clarity with respect to the terms used in the specification and claims.

As used herein, the term “quantitative data” or “quantitative level” or “quantitative amount” refers to data, levels, or amounts associated with any dataset components (e.g., markers, clinical indicia,) that can be assigned a numerical value.

As used herein, the term “subject” refers to animal or mammal. Typically, the terms “subject” and “patient” are used herein interchangeably in reference to a human individual.

As used herein, the term “bodily sample” refers to a sample that may be obtained from a subject (e.g., a human) or from components (e.g., tissues) of a subject. The sample may be of any biological tissue or fluid with which biomarkers described herein may be assayed. Frequently, the sample will be a “clinical sample”, i.e., a sample derived from a patient. Such samples include, but are not limited to, bodily fluids, e.g., urine, blood, plasma, breath, or sera; and archival samples with known diagnosis, treatment and/or outcome history. The term biological sample also encompasses any material derived by processing the biological sample. Processing of the bodily sample may involve one or more of, filtration, distillation, extraction, concentration, inactivation of interfering components, addition of reagents, and the like.

As used herein, the terms “control” or “control sample” refer to one or more biological samples isolated from an individual or group of individuals that are normal (i.e., healthy).

Embodiments described herein relate to a sensor for detecting, identifying, quantifying, and/or determining the amount or level of nitric oxide (NO) in a sample, and particularly relates to a disposable, and cost-effective biosensor for detecting, identifying, quantifying, and/or determining the amount or level of nitric oxide (NO) in a bodily sample or bodily fluid, such as breath, blood, and other physiological fluids.

The sensors described herein utilize an electrochemical method to oxidize nitric oxide and thereby generate electrical currents indicative of the concentration of nitric oxide in a sample, such as a bodily sample. The sensor is configured such that a polyelectrolye film covers or encapsulates portions of a working and counter electrode in a nitric oxide detection region of the sensor. The polyelectrolyte film prevents the substantial diffusion of interfering substances to the electrodes and includes at least one compound that is capable of increasing the rate of electrochemical oxidation-reduction reaction with nitric oxide and providing the detection of nitric oxide at a lower oxidation potential compared to a sensor that does not include the compound.

The sensor may be used in a variety of contexts. Nitric oxide release has been detected in response to both infection and to aseptic tissue injury. In a device intended to provide early diagnosis of infection, it would be important to be able to distinguish between these two conditions. Studies in which oxidation products of nitric oxide have been assessed, show that nitric oxide production was significantly higher in sepsis than in trauma without infection. Thus, the detection of nitric oxide in a bodily sample, such as blood or breadth, should provide non-invasive detection of the onset of infection or inflammation in a subject. Such detection is early enough in the inflammatory process to provide early warning to a medical care provider, thereby allowing for therapeutic treatment of the infection and/or inflammatory response to begin.

In some embodiments, the sensor may be used to identify individuals with probable asthma, particularly very young children with airway inflammation. Early detection of an asthmatic condition may enable a health care provider to appropriately treat the condition before the individual experiences adverse affects in lung function. Research-based evidence suggests that the higher the concentration of nitric oxide (NO) in an individual's exhalation, the more likely the individual is to suffer from an asthmatic condition. Other embodiments may be configured to differentiate between asthma and other conditions that mimic asthma, such as, but not limited to, post-nasal drainage, gastroesophageal reflux, vocal cord dysfunction, and Chronic Obstructive Pulmonary Disease (COPD). In still other embodiments, the sensor described herein may help the clinician determine whether patient's medication regimen needs to be increased or decreased and is useful for monitoring overall control of the condition.

It will be appreciated that the sensor described herein can detect, identify, quantify, and/or determine NO concentration in any liquid and/or gas sample of biologic or non-biologic origin.

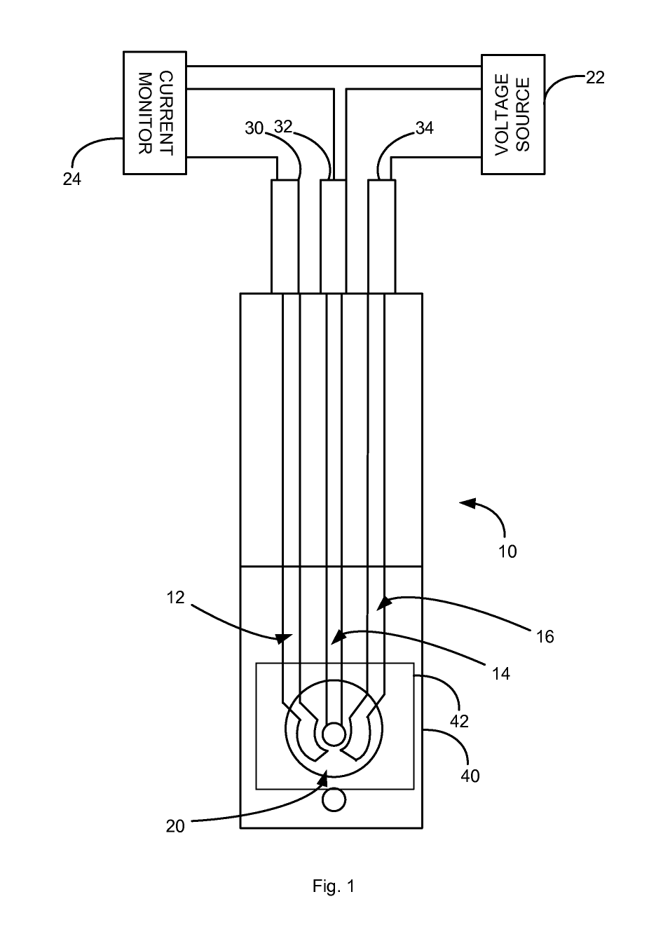

FIG. 1 illustrates a sensor 10 in accordance with an embodiment of the application. The sensor 10 is a three-electrode sensor including a counter electrode 12, a working electrode 14, and a reference electrode 16 that are formed on the surface of a substrate. A dielectric layer 40 covers a portion of the working electrode 12, counter electrode 14 and reference electrode 16. The dielectric layer 40 includes an aperture 20, which defines a detection region of the working electrode 12, counter electrode 14, reference electrode 16 that is exposed to samples in which the levels of nitric oxide are detected. A polyelectrolyte film 42 covers the detection region 20 and exposed portions working electrode, counter electrode, reference electrode.

A voltage source 22 is connected to the working and reference electrodes 14, 16. A current measuring device 24 is connected to the working and counter electrodes 14, 12 to measure the current generated by the redox reaction of nitric oxide when a sample or biological sample contacts the detection region 20 of the sensor 10.

The working electrode 14 is the site of the redox reaction of nitric oxide, and where the charge transfer occurs. The function of the counter electrode 12 is to complete the circuit, allowing charge to flow through the sensor 10. The working electrode 14 and the counter electrode 12 are preferably formed of the same material, although this is not a requirement. Examples of materials that can be used for the working electrode 14 and counter electrode 12 include, but are not limited to, gold, platinum, palladium, silver, carbon, alloys thereof, and composites thereof.

Examples of materials that can be used to form the reference electrode 16 are silver-silver chloride and mercury-mercuric chloride (Calomel). Silver-silver chloride is preferred. The silver can be applied to a substrate in the form of a silver ink, which is commercially available, or can be made using finely dispersed metal particles, solvent, and a binder. Respective silver contact pads 30, 32, and 34 are connected with each of the electrodes 12, 14, and 16. This reference electrode can be thick film printed on the same substrate of the working and counter electrode and also can be used externally.

The polyelectrolyte film covering the detection region and exposed portions working electrode, counter electrode, reference electrode includes a mixture of a perfluorosulfonic acid polymer and at least one metalloporphyrins compound or metallophthalocyanine compound.

The polyelectrolyte film can be selectively permeable or semi-permeable to allow passage of nitric oxide through the film and inhibit passage of anions that interfere with nitric oxide detection. Other substances may also be permitted to diffuse through the film. Such biologically relevant substances include, but are not limited to, nitrogen, oxygen, carbon monoxide, carbon dioxide, and nitrogen dioxide. These biologically relevant substances generally possess different electric potentials than that of nitric oxide and, thus, typically do not interfere with the oxidation of nitric oxide.

In some embodiments, the perfluorosulfonic acid polymer can be a cation permeable thermoplastic perfluorosulfonic acid polymer that can be solution cast to provide a thin film. An example of a thermoplastic perfluorosulfonic acid polymer, which can be solution cast, is Nafion, which is commercially available from Ion-power Inc. (New Castle, Del.). The use of a cation permeable polymer can also minimize any potential interference by any anion.

In some embodiments, the perfluorosulfonic acid polymer can comprise Nafion or a Nafion blend. Nafion can be blended with other other thermoplastic polymers or blends of polymers typically used in forming a polyelectrolyte films. Example of such polymers are sulfonated derivatives of polyphosphazene, poly(ether ketone), polysulfone, polyetetrafluoroethylene, and polyimide.

The at least one metalloporphyrin compound or metallophthalocyanine compound is capable of increasing the rate of electrochemical oxidation-reduction reaction with nitric oxide and providing the detection of nitric oxide at a lower oxidation potential compared to a sensor without the compound. In terms of the practical application, the metalloporphyrins compound or metallophthalocyanine compound can shorten the reaction time and lower the applied electrochemical potential for detection of nitric oxide in a sample, such as a biological sample or bodily fluid. Lowering the applied potential often leads to the minimization of electrochemical oxidation or reduction of other species presented, resulting in a minimization of interference caused by the unwanted reaction of the confounding species. As a result, a highly specific biosensor can be obtained and produced.

In other embodiments, the at least one metalloporphyrin compound or metallophthalocyanine compound contains as central atoms a metal atom selected from Fe, Co, Ni, Zn, Mn, Cu, Ru, V, Pb, or Cr. The at least one metalloporphyrin compound or metallophthalocyanine compound can be provided in the polyelectrolyte film at an amount of about 0.001% to about 90% by weight of the polyelectrolyte film, for example, about 0.01% to about 50% by weight, or about 0.01% to about 10% by weight. In some embodiments, the at least one metalloporphyrin comprises nickel(II)poly-tetrakis(3-methoxy-4-hydroxy-phenyl) porhydrin.

The voltage source can apply a voltage potential to the working electrode 14 and reference and/or counter electrode 16, 12, depending on the design of the sensor 10. The current between the working electrode 14 and counter electrode 16 can be measured with a measuring device or meter. Such current is due to the reduction occurring at the working electrode 12 of nitric oxide in the sample that is provided at the detection region.

The amount or level of current measured is proportional to the level or amount of nitric oxide in the sample. In some embodiments, where the sample is a bodily sample obtained from a subject that has or is suspected of having a condition, pathology, or disorder associated with aberrant nitric oxide levels, once the current level generated by the bodily sample tested with the sensor is determined, the level can be compared to a predetermined value or control value to provide information for diagnosing or monitoring of the condition, pathology, or disorder in a subject. For example, the current level can be compared to a predetermined value or control value to determine if a subject has an infection. An increased current level compared to a predetermined value or control value can be indicative of the subject having infection; whereas similar or decreased current level compared to a predetermined value or control value can be indicative of the absence of infection in the subject

In other embodiments, the current level generated by the bodily sample obtained from the subject can be compared to a current level of a bodily sample previously obtained from the subject, such as prior to administration of a therapeutic. Accordingly, the methods described herein can be used to measure the efficacy of a therapeutic regimen for the treatment of a condition, pathology, or disorder associated with aberrant nitric oxide levels in a subject by comparing the current level obtained before and after a therapeutic regimen. Additionally, the methods described herein can be used to measure the progression of a condition, pathology, or disorder associated with aberrant nitric oxide levels in a subject by comparing the current level in a bodily sample obtained over a given time period, such as days, weeks, months, or years.

The current level generated by a bodily sample of the subject may also be compared to a predetermined value or control value to provide information for determining the severity or aggressiveness of a condition, pathology, or disorder associated with aberrant nitric oxide levels in the subject. A predetermined value or control value can be based upon the current level in comparable samples obtained from a healthy or normal subject or the general population or from a select population of control subjects.

The predetermined value can take a variety of forms. The predetermined value can be a single cut-off value, such as a median or mean. The predetermined value can be established based upon comparative groups such as where the current level in one defined group is double the current level in another defined group. The predetermined value can be a range, for example, where the general subject population is divided equally (or unequally) into groups, or into quadrants, the lowest quadrant being subjects with the lowest current level, the highest quadrant being individuals with the highest current level. In an exemplary embodiment, two cutoff values are selected to minimize the rate of false positive and negative results.

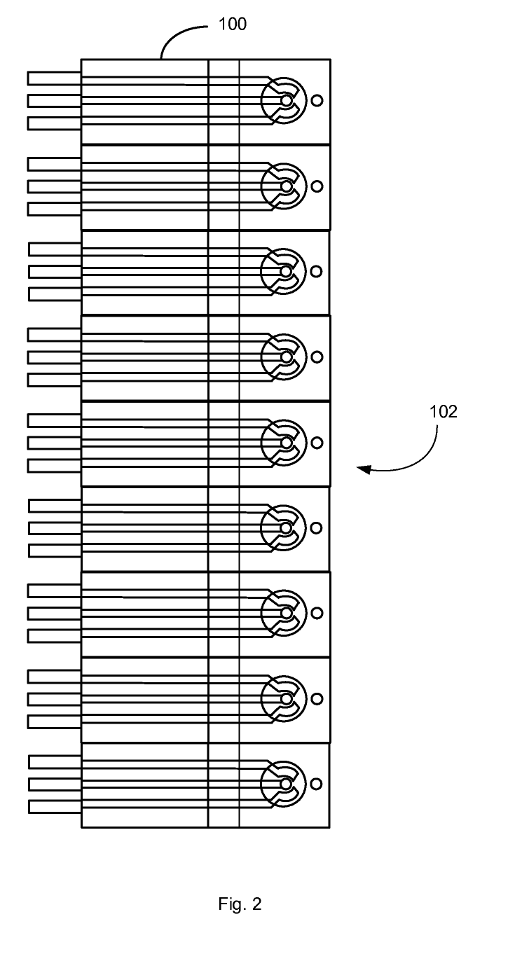

The biosensor illustrated in FIGS. 1 and 2 can be fabricated on a substrate 100 formed from polyester or other electrically non-conductive material, such as other polymeric materials, alumina (Al₂O₃), ceramic based materials, glass or a semi-conductive substrate, such as silicon, silicon oxide and other covered substrates. Multiple sensor devices 102 can thus be formed on a common substrate 100 (FIG. 2). As will be appreciated, variations in the geometry and size of the electrodes are contemplated.

The biosensor can be made using a thin film, thick film, and/or ink-jet printing technique, especially for the deposition of multiple electrodes on a substrate. The thin film process can include physical or chemical vapor deposition. Electrochemical sensors and thick film techniques for their fabrication are discussed in U.S. Pat. No. 4,571,292 to C. C. Liu et al., U.S. Pat. No. 4,655,880 to C. C. Liu, and co-pending application U.S. Ser. No. 09/466,865, which are incorporated by reference in their entirety. By way of example, in the case of the carbon electrodes, active carbon is mixed with a binder, deposited like an ink on the substrate, and allowed to dry.

In some embodiments, the working electrode, counter electrode, and reference electrode may be formed using laser ablation, a process which can produce elements with features that are less than one-thousandth of an inch. Laser ablation enables the precise definition of the working electrode, counter electrode, and reference electrode as well as electrical connecting leads and other features, which is required to reduce coefficient of variation and provide accurate measurements. Metalized films, such as Au, Pd, and Pt or any metal having similar electrochemical properties, that can be sputtered or coated on plastic substrates, such as PET or polycarbonate, or other dielectric material, can be irradiated using laser ablation to provide these features.

In one example, a gold film with a thickness of about 300 to about 2000 A can be deposited by a sputtering technique resulting in very uniform layer that can be laser ablated to form the working and counter electrodes. The counter electrode can use other materials. However, for the simplicity of fabrication, using identical material for both working and counter electrodes will simplify the fabrication process providing the feasibility of producing both electrodes in a single processing step. An Ag/AgCl reference electrode, the insulation layer, and the electrical connecting parts can then be printed using thick-film screen printing technique.

In some embodiments, the overall dimensions of an individual sensors are chosen to be 33.0×8.0 mm². The total width of each individual biosensor is approximately 2.8 mm with a working electrode of 1.0 mm in diameter sufficiently to accommodate up to a 5 μL sample volume. These sizes can be changed as needed.

The polyelectrolyte film can then be provided on the sensor so formed. The first step in providing the film on the sensor is to clean the sensor electrode surface minimizing any oxide or dirt layer on the surface. Parafilm can be cut and used cover the reference electrode. Any other means to cover and protect the reference electrode can also be used. The sensor can then be rinsed with 1:1 v/v ratio ethanol and deionized water solution and dried by pure nitrogen gently.

A polyelectrolyte film can then be applied to the sensor. In some embodiments, the polyelectrolyte film that is applied to sensor includes a mixture of Nafion and Ni(II)-TMHPP (nickel(II)-tetrakis(3-methoxy-4-hydroxy-phenyl) porphyrin). For example, 5 grams of Nafion solution (# D2020, Ion Power Inc., Bear, Del.) is used which contained 15 wt % of Nafion in 85 wt. % of alcohol. The Nafion solution is then diluted with di-ethylene glycol or ethylene glycol. 1.125 g. of ethylene glycol can be added to the solution resulting a total weight of 6.125 g. This mixture is then placed in a vacuumed oven and heated to 80 C in order to vaporize the alcohol. At the end of approximately 2 hours, the total mixture is about 2.25 g. which translated into approximately 2.25 mL. (the density of the mixture is about 1 g./mL) and served as a Nafion-based solution for the incorporation of Ni(II)-TMHPP. Ni(II)-TMHPP (cat#T40794 Frontier Scientific, Cogan, Utah) is then added into the prepared Nafion solution. 0.300 g. of the Nafion solution can then mixed with 0.0010 g. of Ni(II)-TMHPP. The ratio of the Ni-TMHPP and Nafion mixture however can vary and any ratio is potentially applicable for use herein.

In one example, about 5 μL of the mixed Nafion and Ni(II)-TMHPP can be placed on top of the sensor, so that the solution spreads evenly over the whole sensor. The sensor can then be dried either at ambient condition overnight or dried in a vacuumed oven at 80C. The dried biosensor is then ready for use.

By way of example, in the preliminary testing of this sensor, a small plastic test chamber with gas inlet and outlet is constructed and used. The overall dimensions of the test box are 13.3 cm×5.7 cm×5.7 cm. The dimensions of this chamber are not critical in this evaluation process, and they can be varied. A mass-flow controlled system with two gas inlets are connected to the test box. A gas mixture of 225 ppm of NO in N₂ and a pure air are used and controlled producing the required concentration of NO in the test box. The gas mixture first passes through a glass gas bubbler humidifying the gas prior to feed into the test box. The test gas also can be fed directly into the test chamber without the gas humidifier. The gas mixture with known NO concentration passes through the test box for two minutes in order to have a fixed, equilibrated nitric oxide concentration in the test box. This two minute of time is not a critical parameter. In this preliminary test, it is sufficient to provide a well defined nitric oxide concentration in the test environment. Then both gas inlet and outlet valves are then closed, and electrochemical measurement is then made. An Electrochemical Workstation (CHI 660 Model A to D, Austin, Tex.) is used in these measurements, and any other electrochemical potentiostat can also be employed. Differential pulse voltammetry (DPV) is employed as the electrochemical analytical technique for this measurement.

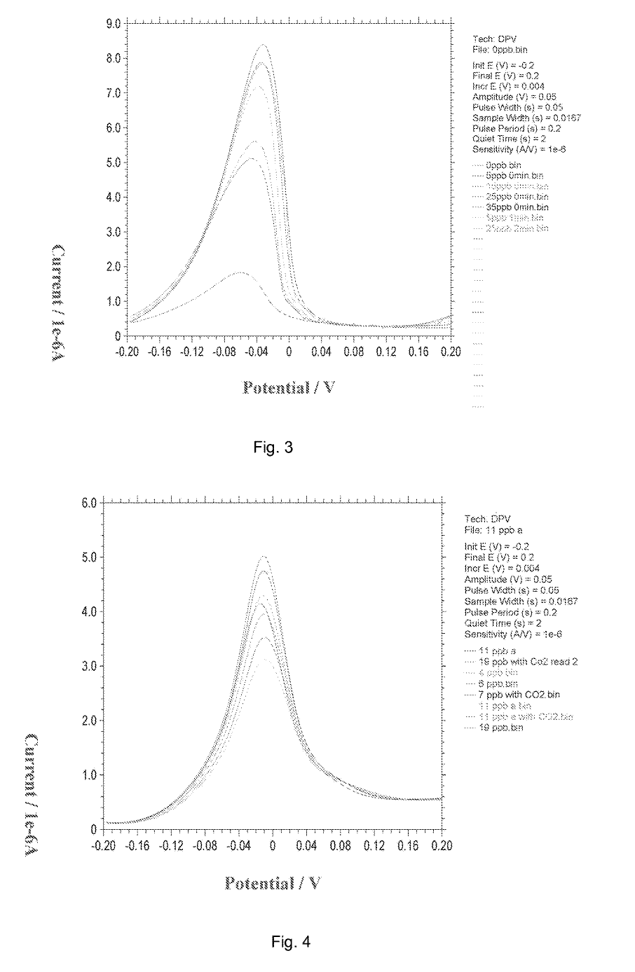

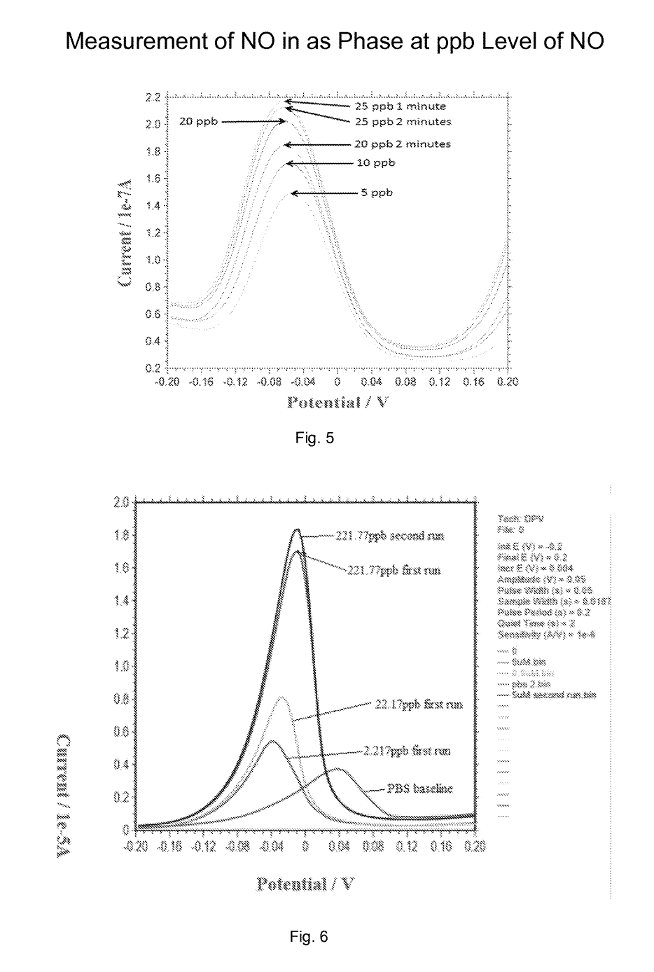

In order to carry out the detection of NO in ppb (parts per billion) range, a different experimental arrangement is needed. NO gas of 250 ppb level is used. The NO gas is then mixed with air further diluting the NO concentration in 4-25 ppb range. A Nitric Oxide Analyzer (GE Siever 280 System) is used to provide an independent NO concentration measurement. The air diluted NO gas sample is then used to fill a balloon type gas reservoir. The gas from the balloon is then hand-pressed feeding into the test chamber. Differential pulse voltammetric measurement and/or cyclic voltammetric measurements can then be made. It requires about 20 seconds completing one experimental run. This operating time can be adjusted, if needed. FIGS. 3, 4, and 5 show the typical experimental response to NO concentration in air in ppb level for both gold electrode (FIG. 3) and platinum electrode (FIGS. 4 and 5) sensors. FIG. 5 shows the concentration of NO in ppb detected in various gas samples using the sensor.

In another example, the sensor was used to detect NO concentration in liquid samples, such as phosphate buffered saline (PBS). In this example, the sensor was used to measure NO concentration in PBS samples with varying concentrations of NO. FIG. 6 shows the concentration of NO in ppb detected in the various PBS samples using the sensor.

From the above description of the invention, those skilled in the art will perceive improvements, changes and modifications. Such improvements, changes and modifications within the skill of the art are intended to be covered by the appended claims. All references, publications, and patents cited in the present application are herein incorporated by reference in their entirety.

Having described the invention the following is claimed:

1. A sensor for the detection of nitric oxide comprising:
a substrate;
a working electrode formed on a surface of the substrate;
a counter electrode formed on the surface of the substrate;
a dielectric layer covering a portion of the working electrode and counter electrode and defining an aperture exposing other portions of the working electrode and counter electrode; and
a polyelectrolyte film covering the exposed portions working electrode and counter electrode, the polyelectrolyte film including at least one metalloporphyrin compound or metallophthalocyanine compound capable of increasing the rate of electrochemical oxidation-reduction reaction with nitric oxide and provides the detection of nitric oxide at a lower oxidation potential.

2. The sensor of claim 1, wherein the polyelectrolyte film is semi-permeable to allow passage of nitric oxide through the film and inhibit passage anions that interfere with nitric oxide detection.

3. The sensor of claim 1, wherein the polyelectrolyte film comprises a mixture of a perfluorosulfonic acid polymer and at least one metalloporphyrin compound or metallophthalocyanine compound.

4. The sensor of claim 3, wherein the perfluorosulfonic acid polymer comprises Nafion or a Nafion blend.

5. The sensor of claim 5, wherein the at least one metalloporphyrin compound or metallophthalocyanine compound contains as central atoms a metal atom selected from Fe, Co, Ni, Zn, Mn, Cu, Ru, V, Pb, or Cr.

6. The sensor of claim 3, wherein the at least one metalloporphyrin compound or metallophthalocyanine compound is provided in the polyelectrolyte film at an amount of about 0.01 to about 10% by weight of the polyelectrolyte film.

7. The sensor of claim 5, wherein the at least one metalloporphyrin comprises nickel(II)poly-tetrakis(3-methoxy-4-hydroxy-phenyl) porhydrin.

8. The sensor of claim 1, wherein the working electrode and the counter electrode comprise metalized films.

9. The sensor of claim 1, wherein the working electrode and counter electrode independently comprise gold, platinum, palladium, silver, carbon, alloys thereof, and composites thereof.

10. The sensor of claim 9, the metalized films are provided on the surface of the substrate by sputtering or coating the films on the surface and wherein the working electrode and the counter electrode are formed using laser ablation

11. The sensor of claim 1, further comprising a reference electrode on the surface of the substrate, the dielectric covering a portion of the reference electrode.

12. The sensor of claim 1, further comprising a measuring device for applying voltage potentials to the working electrode and counter electrode and measuring the current flow between the working electrode and counter electrode.

13. A biosensor for detection nitric oxide in a bodily sample, the biosensor comprising:
a substrate;
a working electrode formed on a surface of the substrate;
a counter electrode formed on the surface of the substrate;
a reference electrode formed on the surface of the substrate;
a dielectric layer covering a portion of the working electrode, counter electrode, and reference electrode, the dielectric defining an aperture exposing other portions of the working electrode, counter electrode, and reference electrode; and
a cation permeable polyelectrolyte film covering the exposed portions of the working electrode, counter electrode, and reference electrode, the polyelectrolyte film including a mixture of a perfluorosulfonic acid polymer and at least one metalloporphyrin compound or metallophthalocyanine compound capable of increasing the rate of electrochemical oxidation-reduction reaction with nitric oxide and provides the detection of nitric oxide at a lower oxidation potential.

14. The sensor of claim 14, wherein the perfluorosulfonic acid polymer comprises Nafion or a Nafion blend.

15. The sensor of claim 13, wherein the at least one metalloporphyrin compound or metallophthalocyanine compound contains as central atoms a metal atom selected from Fe, Co, Ni, Zn, Mn, Cu, Ru, V, Pb, or Cr.

16. The sensor of claim 13, wherein the at least one metalloporphyrin compound or metallophthalocyanine compound is provided in the polyelectrolyte film at an amount of about 0.01 to about 10% by weight of the polyelectrolyte film.

17. The sensor of claim 13, wherein the at least one metalloporphyrin comprises nickel(II)poly-tetrakis(3-methoxy-4-hydroxy-phenyl) porhydrin.

18. The sensor of claim 13, wherein the working electrode and the counter electrode comprise metalized films.

19. The sensor of claim 13, wherein the working electrode and counter electrode independently comprise gold, platinum, palladium, silver, carbon, alloys, thereof and composites thereof.

20. The sensor of claim 18, the metalized films are provided on the surface of the substrate by sputtering or coating the films on the surface and wherein the working electrode and the counter electrode are formed using laser ablation

21. The sensor of claim 1, further comprising a measuring device for applying voltage potentials to the working electrode and counter electrode and measuring the current flow between the working electrode and counter electrode.

22. The sensor of claim 13, wherein the biological sample is a biological fluid.

23. The sensor of claim 22, wherein the biological fluid is exhaled air, blood, or other physiological fluids.
